SEQUENCE LISTING (33 sequences)

SEQ ID NO: 1 - DNA, 1487 bp, synthetic construct
  source 1..1487: mol_type = other DNA
atatttcttaaaaatattgaaatataattactttccctattcatttgttgtgtcccaaaatgcgtaataaacatgctttaagtagcataaaaggctgtattaaaatcttgactagtcttacaagttagtcatctgattttttttttctttaagaaaatttttttttttttttttattcagttacataaatgcatagattcgtctaagctgaatccaaaaataaggttcattaccctttaactgctagttggtcagacttgcgattaacacactttcttaacatagagactagtttatgcaactaatttaaatgatcattacaaattatcatatttccataagagtgttaaatttgatcactaaattaatatcagccattcctaaaggctgatatgagcaagtgatcacagtcaaacccccaaacatttggtatctctgaaaagtactctttacaggacatccagacatgtgagatctcttcacactctaaccaaatttcatcttgccacgacagatcatttcacagtcaaaatacattaaaactatcagaacatttcataaatgtctgacatcatgtcattctagcaaaggttttcaccaaacaaacacacttagtcatgtcaaagcagctttacagtgtcagtagaaaaatggtttgtcaataatgcaagaaaacaaactcctcaacatttctgctctggccaaatgctatttgtgattatgatgggttaaatgcagcagaccaggactttcaccttcacctcaccagtcactcaacactttcacaaccctaagatcaaaacagcaggaagaaagaatggccatttgcatagcgaatgataacaattcaaggtcattcattcacatgcttttaaaacatgcagagacactctcacattcattctgttgccatgtcttctaagagattgatgcagaaatttgaggaactgaacgcttgtcttccccagaagaaaagaagaacacagcttgaagttctttgtggagcaattgaccacatcacatttctgacaaatttattaaaagggtcagtttaaacgattaataataaaattgttataatatcatttaattattttatgcaatttattaagcagcatttgatattgtttatgtattctgctgatcagttgcacaaatgtgtttggtgtttataggcatggatctggatctgatggacgtgtggagagagaggacggaccggcagaacatgctctggcaaacgctgctgagaaggagccagatggagtgtaacttcagccctgcagaacatggttttaactgagagtctcacagttcatctgtatttctctatagtctccactacacacatgtcttctgccacgctgaggtgtgaaccctaaatatgtgtctgcttttgctgagtaaaaataaactttctgaagcatcacctgtgtgtgtttcattctgtttgtatgatcatactttctttacaaaaattcttcaca

SEQ ID NO: 2 - DNA, 231 bp, synthetic construct
  source 1..231: mol_type = other DNA
atgtcttctaagagattgatgcagaaatttgaggaactgaacgcttgtcttccccagaagaaaagaagaacacagcttgaagttctttgtggagcaattgaccacatcacatttctgacaaatttattaaaagggcatggatctggatctgatggacgtgtggagagagaggacggaccggcagaacatgctctggcaaacgctgctgagaaggagccagatggagtgtaa

SEQ ID NO: 3 - protein, 76 aa, synthetic construct
MSSKRLMQKFEELNACLPQKKRRTQLEVLCGAIDHITFLTNLLKGHGSGSDGRVEREDGPAEHALANAAEKEPDGV

SEQ ID NO: 4 - DNA, 73 bp, synthetic construct
  source 1..73: mol_type = other DNA
atgtcttctaagagacggaccggcagaacatgctctggcaaacgctgctgagaaggagccagatggagtgtaa

SEQ ID NO: 5 - DNA, 3131 bp, synthetic construct
  source 1..3131: mol_type = other DNA
cttcattgtgcagatgcctacattatataactttataactttgttgactaaaatttagctgaaactacaaaacaccaaaatgcagtaaattatctgaaacttaaaaaaattgataaaactgaaataaaacaaaattaaaaattagttttttttttcttgcacccagaaccaacatgcaggatggaaaattgtgaaaattttgggccattgccattaaatgtatgaaacacacacagacacacacacactgagaggaagaaaacaaaaacagctttgatgatgtttgaggatattaaccaaatcaatcagcacatgttcattcatatctctatgaattctgccgctatcatagccctgaccacagacctcagggactttgagcatctctctctcttttttgcattatcacttttcactcccagcagaaggctagtatcaggctttttttttttcttttctctctctctctctctctctctgaatgtgtctctgaaaagctttcatgtcattacattgtttagagaaattagaaaaataacttttattgagaaattatatttgattcatgtcacgtcttttgtagagaactccagaactcataatgatatctaatatttcttaaaaatattgaaatataattactttccctattcatttgttgtgtcccaaaatgcgtaataaacatgctttaagtagcataaaaggctgtattaaaatcttgactagtcttacaagttagtcatctgattttttttttctttaagaaaatttttttttttttttttattcagttacataaatgcatagattcgtctaagctgaatccaaaaataaggttcattaccctttaactgctagttggtcagacttgcgattaacacactttcttaacatagagactagtttatgcaactaatttaaatgatcattacaaattatcatatttccataagagtgttaaatttgatcactaaattaatatcagccattcctaaaggctgatatgagcaagtgatcacagtcaaacccccaaacatttggtatctctgaaaagtactctttacaggacatccagacatgtgagatctcttcacactctaaccaaatttcatcttgccacgacagatcatttcacagtcaaaatacattaaaactatcagaacatttcataaatgtctgacatcatgtcattctagcaaaggttttcaccaaacaaacacacttagtcatgtcaaagcagctttacagtgtcagtagaaaaatggtttgtcaataatgcaagaaaacaaactcctcaacatttctgctctggccaaatgctatttgtgattatgatgggttaaatgcagcagaccaggactttcaccttcacctcaccagtcactcaacactttcacaaccctaagatcaaaacagcaggaagaaagaatggccatttgcatagcgaatgataacaattcaaggtcattcattcacatgcttttaaaacatgcagagacactctcacattcattctgttgccatgtcttctaagagattgatgcagaaatttgaggaactgaacgcttgtcttccccagaagaaaagaagaacacagcttgaagttctttgtggagcaattgaccacatcacatttctgacaaatttattaaaagggtcagtttaaacgattaataataaaattgttataatatcatttaattattttatgcaatttattaagcagcatttgatattgtttatgtattctgctgatcagttgcacaaatgtgtttggtgtttataggcatggatctggatctgatggacgtgtggagagagaggacggaccggcagaacatgctctggcaaacgctgctgagaaggagccagatggagtgtaacttcagccctgcagaacatggttttaactgagagtctcacagttcatctgtatttctctatagtctccactacacacatgtcttctgccacgctgaggtgtgaaccctaaatatgtgtctgcttttgctgagtaaaaataaactttctgaagcatcacctgtgtgtgtttcattctgtttgtatgatcatactttctttacaaaaattcttcacaaatatttttgacaggtggtttataatgattaaatgcttcaaacaatatataatgcttaatagatcattaatagattctaatagctacgagacaatatccttgttaaagtttaaagattaacacataattatttaatgtaaacaaaatgtataaacaccaagctataaaacaccaagctgcaattctattaaattaagagtttatatcctacagtttgagtttttatgcaagttataatgttatgacataaaaaaactattaatactgtaatgagagttagctgacatgtagttgcacaaaaatgctattttaaatattttgagaggactaacaaaacatgtaaaatagcataaatggtttggtttgtgggttcttaatgttgaccatttcccaaatatgttcagctaattctagaattaattctagtttaggacatttgtatcagaatggtgcatgataacagcatgttagaaaactattactactgaagaaaaagatgatagtgagataagtgtgattatatgctattatgatttcagcctcagggttcagaaacccacatttccttcattttatcaaacattttgatataaactcacatcttctgtacgaaattaagtatgcttcactctgtatgtctgaatcacgtgtcaaatacatctgaaagctaagaacatagccaatttacacttacatttatttagcagacgctttttatccaaagcgacaatacagtaaaataataaaaaacacaatacttatcacaaaaaaaaaattatgaacacaaataaatattataaatatttatgaatacagcaaaacataaagcatttcacaatttaatcatgcaaacacatttcccaatgtaaacaattataagatactactgatatttaaaatatctggtgaatgacaagaatctttcaaaccaaatctgacgttaagccaaaatatctaataataatcataataataattacagattaaaattcacctccagtctatacactacagagtcatcaaatcgatacca

SEQ ID NO: 6 - DNA, 224 bp, synthetic construct
  source 1..224: mol_type = other DNA
atgtcttctaagagattgatgcagaaatttgaggaactgaacgcttgtcttccccagaagaaaagaagaacacagcttgaagttctttgtggagcaattgaccacatcacatttctgacaaatttattaaaagggcatggatctggatctgatggacgtgtggagagagaccggcagaacatgctctggcaaacgctgctgagaaggagccagatggagtgtaa

SEQ ID NO: 7 - DNA, 25 bp, synthetic construct
  source 1..25: mol_type = other DNA
tggtatcgatttgatgactctgtag

SEQ ID NO: 8 - DNA, 25 bp, synthetic construct
  source 1..25: mol_type = other DNA
cttcattgtgcagatgcctacatta

SEQ ID NO: 9 - DNA, 25 bp, synthetic construct
  source 1..25: mol_type = other DNA
aactgctagttggtcagacttgcga

SEQ ID NO: 10 - DNA, 25 bp, synthetic construct
  source 1..25: mol_type = other DNA
cacatatttagggttcacacctcag

SEQ ID NO: 11 - DNA, 20 bp, synthetic construct
  source 1..20: mol_type = other DNA
agaacatgctctggcaaacg

SEQ ID NO: 12 - DNA, 21 bp, synthetic construct
  source 1..21: mol_type = other DNA
tttagggttcacacctcagcg

SEQ ID NO: 13 - DNA, 19 bp, synthetic construct
  source 1..19: mol_type = other DNA
atggagtggaacggaaggt

SEQ ID NO: 14 - DNA, 23 bp, synthetic construct
  source 1..23: mol_type = other DNA
cagacaaatgagaacaagaggaa

SEQ ID NO: 15 - DNA, 21 bp, synthetic construct
  source 1..21: mol_type = other DNA
gatgatgaaattgccgcactg

SEQ ID NO: 16 - DNA, 22 bp, synthetic construct
  source 1..22: mol_type = other DNA
accaaccatgacaccctgatgt

SEQ ID NO: 17 - DNA, 25 bp, synthetic construct
  source 1..25: mol_type = other DNA
tgacaaatttattaaaaaggcatgg

SEQ ID NO: 18 - DNA, 25 bp, synthetic construct
  source 1..25: mol_type = other DNA
cacatatttagggttcacacctcag

SEQ ID NO: 19 - DNA, 19 bp, synthetic construct
  source 1..19: mol_type = other DNA
ggccagagcagaaatgttg

SEQ ID NO: 20 - DNA, 20 bp, synthetic construct
  source 1..20: mol_type = other DNA
ggacgtgtggagagagagga

SEQ ID NO: 21 - DNA, 20 bp, synthetic construct
  source 1..20: mol_type = other DNA
ggcagaagacatgtgtgtag

SEQ ID NO: 22 - DNA, 55 bp, synthetic construct
  source 1..55: mol_type = other DNA
tgtaatacgactcactataggccagagcagaaatgttggttttagagctagaaat

SEQ ID NO: 23 - DNA, 56 bp, synthetic construct
  source 1..56: mol_type = other DNA
tgtaatacgactcactataggacgtgtggagagagaggagttttagagctagaaat

SEQ ID NO: 24 - DNA, 56 bp, synthetic construct
  source 1..56: mol_type = other DNA
tgtaatacgactcactataggcagaagacatgtgtgtaggttttagagctagaaat

SEQ ID NO: 25 - DNA, 25 bp, synthetic construct
  source 1..25: mol_type = other DNA
aaaaaaagcaccgactcggtgccac

SEQ ID NO: 26 - DNA, 25 bp, synthetic construct
  source 1..25: mol_type = other DNA
cacatatttagggttcacacctcag

SEQ ID NO: 27 - DNA, 43 bp, synthetic construct
  source 1..43: mol_type = other DNA
tcgataagcttgatatcgtggtatcgatttgatgactctgtag

SEQ ID NO: 28 - DNA, 43 bp, synthetic construct
  source 1..43: mol_type = other DNA
atacgactcactatagttcttcattgtgcagatgcctacatta

SEQ ID NO: 29 - DNA, 25 bp, synthetic construct
  source 1..25: mol_type = other DNA
aactgctagttggtcagacttgcga

SEQ ID NO: 30 - DNA, 25 bp, synthetic construct
  source 1..25: mol_type = other DNA
atgaactggattttctgcaacactg

SEQ ID NO: 31 - DNA, 24 bp, synthetic construct
  source 1..24: mol_type = other DNA
aaagtatctggctagaatcttact

SEQ ID NO: 32 - DNA, 25 bp, synthetic construct
  source 1..25: mol_type = other DNA
tgttgagtgactggtgaggtgaagg

SEQ ID NO: 33 - RNA, 447 nt, synthetic construct
  source 1..447: mol_type = other RNA
atgcagagacactctcacattcattctgttgccatctcttctaagagattgatgcagaaatttgaggaactgaacgcttgtcttccccagaagaaaagaagaacacagcttgaagttctttgtggagcaattgaccacatcacatttctgacaaatttattaaaagggcatggatctggatctgatggacgtgtggagagagaggacggaccggcagaacatgctctggcaaacgctgctgagaaggagccagatggagtgtaacttcagccctgcagaacatggttttaactgagagtctcacagttcatctgtatttctctatagtctccactacacacatgtcttctgccacgctgaggtgtgaaccctaaatatgtgtctgcttttgctgagtaaaaataaactttctgaagcatcaaaaaaaaaaaaaaaaaaaaaaaaaaa

(19) United States
(12) Patent Application Publication
(10) Pub. No.: US 2026/0297672 A1
(43) Pub. Date: Oct. 1, 2026

(54) COMMON CARP SEX-DETERMINING GENE CCMD AND ITS APPLICATIONS IN CARP SEX IDENTIFICATION OR SEX CONTROL

(71) Applicant: Institute of Hydrobiology, Chinese Academy of Sciences, Wuhan (CN)

(72) Inventors: Wei HU, Wuhan (CN); Kuangxin CHEN, Wuhan (CN); Ji CHEN, Wuhan (CN); Binbin TAO, Wuhan (CN); Yanlong SONG, Wuhan (CN); Zuoyan ZHU, Wuhan (CN)

(21) Appl. No.: 19/680,923

(22) Filed: May 18, 2026

Related U.S. Application Data
(63) Continuation of application No. PCT/CN2023/132747, filed on Nov. 20, 2023.

(51) Int. Cl. C12Q 1/6879; A01K 67/0276; C12N 9/22; C12N 15/11; C12N 15/85; C12N 15/90

(52) U.S. Cl. CPC C12Q 1/6879; A01K 67/0276; C12N 9/226; C12N 15/11; C12N 15/8509; C12N 15/902; A01K 2227/40; C12N 2310/20; C12Q 2600/158

(57) ABSTRACT

Common carp sex-determining gene ccMD is used in common carp sex identification and sex control, where the gene is isolated and cloned from common carp and the protein encoded is shown in SEQ ID NO: 3. By utilizing the sequence features of the gene to design specific primers, XX, XY and YY common carp can be rapidly and accurately identified. With the gene serving as a target gene, genetically male but physiologically female common carp, or genetically female but physiologically male common carp, can be rapidly obtained by gene editing technology and gene transfer technology without hormone treatment. The invention uses an accurate genetic target for sex identification and sex-controlled breeding of common carp and provides novel genetic resources for superior germplasm creation in common carp aquaculture and ornamental breeding, as well as for the prevention and control of common carp as invasive alien species.

### CROSS-REFERENCE TO RELATED APPLICATIONS

The subject application is a continuation of PCT/CN2023/132747 filed on Nov. 20, 2023. The contents and subject matter of the PCT international application are incorporated herein by reference.

### REFERENCE TO AN ELECTRONIC SEQUENCE LISTING

The contents of the electronic sequence listing (Name of the File: SequenceListing8032wh.xml; Size: 35,929 Bytes; and Date of Creation: May 16, 2026) is herein incorporated by reference in its entirety.

### TECHNICAL FIELD

The present invention pertains to the field of molecular biology, and more particularly relates to the isolation and cloning of a transcriptional regulatory factor ccMD from common carp (Cyprinus carpio), as well as applications of the gene in common carp sex identification, sex-controlled breeding, and prevention and control of invasive alien species. By utilizing the gene provided by the present invention as a target sequence, the sex of carp (different strains of the genus Cyprinus) can be rapidly and accurately identified, and mutation or overexpression of the ccMD gene can be performed by conventional methods in the art, thereby accurately achieving common carp sex control.

### BACKGROUND ART

Existing fish species account for more than half of all vertebrates, and the family Cyprinidae represents the largest group of freshwater fishes worldwide, comprising approximately 2,420 species. The family consists of 12 subfamilies, including Leuciscinae, Cultrinae, Barbinae, Cyprininae, and Hypophthalmichthyinae, and plays a pivotal role in the origin of species and the evolution of biological systems. To date, approximately 20 sex-determining genes have been identified in fishes; however, no sex-determining gene has been identified in Cyprinidae thus far (Chen et al., 2022).

Common carp is one of the representative species of Cyprinidae. The subfamily Cyprininae comprises Cyprinus, Carassius, Puntioplites, Procypris and Carassioides, among others. The identification of common carp sex-determining genes is of great significance for elucidating sex-determining genes and sex-determination mechanisms in Cyprininae and even throughout Cyprinidae.

Common carp exhibits obvious sexual growth dimorphism, with female carp exhibiting a growth rate approximately 30% higher than that of male carp; therefore, sex-controlled breeding of all-female common carp possesses important application value. Common carp employs an XX/XY sex-determination system. Traditional all-female common carp breeding methods require the use of gynogenetic development to obtain XX female carp with known genetic sex, whereupon the XX gynogenetic female carp are continuously fed hormone-containing feed for three months prior to sexual differentiation to obtain XX pseudo-male carp with reversed sex. Upon sexual maturation in the following year, the XX pseudo-male common carp are crossed with normal female common carp to produce all-female offspring. The method is not only complicated and time-consuming but also requires the administration of hormones during the breeding process of pseudo-male common carp, which may compromise aquatic environmental safety. More importantly, upon cessation of hormone treatment, XX pseudo-male common carp typically undergo sexual reversal back to female, or exhibit significantly compromised spermatogenic capacity, which is unfavorable for industrial-scale production. Through the identification of common carp sex-determining genes and the precise manipulation thereof by gene editing technology, accurate and efficient sex control in common carp can be achieved, which possesses broad application prospects.

Common carp is also listed among the world's 100 most threatening invasive alien species (Luque et al., 2014). In several regions of the Americas, Oceania, and Africa, particularly in the United States, Canada, and Australia, common carp, as an invasive alien species, is widely distributed in major lakes and streams, causing severe damage to local aquatic communities and ecosystems (Zambrano et al., 2006; Weber and Brown, 2011; Forsyth et al., 2013; Bajer and Sorensen, 2015; Vilizzi et al., 2015; Bajer et al., 2016). Currently, invasive carp populations are primarily controlled by artificial removal methods such as water level manipulation, enclosure, electrofishing, and whole-lake poisoning; however, in most cases, the elimination of carp invasion by these methods is impractical (Gutierrez and Teem, 2006; Britton et al., 2011; Vilizzi, 2012; Koehn et al., 2016). Population sex ratio represents a key indicator for regulating population size. In 2006, Gutierrez and Teem proposed a novel biological invasion control strategy termed the “Trojan Y chromosome” approach, wherein YY males are periodically released into specific waters to reduce the proportion of females, thereby achieving accurate and targeted biological management through the regulation of sex ratios in invasive fish populations. The strategy is considered the most promising biological prevention and control approach for invasive fish possessing an XX/XY genetic sex-determination system (Wang et al., 2016). The production process of YY male common carp involves a series of procedures including sequential mating, genetic sex identification, and artificial induction of sex reversal. Theoretically, by crossing XY pseudo-female common carp obtained through artificial induction of sex reversal with wild XY male common carp, a progeny population comprising ¼ XX, 2/4 XY, and ¼ YY individuals can be obtained. Subsequently, the ¼ YY proportion of the progeny can be subjected to estrogen feeding to induce sex reversal, thereby obtaining a YY pseudo-female common carp population. However, under conventional conditions, effective discrimination among XX, XY, and YY carp at the juvenile stage is impossible. Upon sexual maturation of XY pseudo-female carp and candidate YY carp, test crosses are performed between XY pseudo-female common carp and wild XY male common carp, as well as between candidate YY common carp and XX female common carp, whereupon the sexes of the respective offspring are analyzed to verify their genetic sex types. The process not only consumes substantial manpower and material resources but also requires an extended duration. To date, no successful induction of YY pseudo-female common carp has been reported. Therefore, the development of molecular techniques capable of rapidly and accurately distinguishing and identifying XX, XY, and YY common carp is urgently required. Common carp possessing an XX/XY sex-determination system, the male sex-determining gene constitutes a unique gene located on the Y chromosome. Rapid identification of XX, XY, and YY common carp can be accurately and specifically achieved by amplifying the sequence of the sex-determining gene and quantifying copy number differences. Accordingly, the identification of common carp sex-determining genes possesses important practical application value for the rapid and efficient identification of YY common carp and the prevention and control of biological invasion by common carp.

Sex-specific molecular marker-assisted breeding of Cyprinus common carpio var. rubrofuscus also possesses important application value for ornamental Cyprinus carpio var. rubrofuscus cultivation. Female Cyprinus carpio var. rubrofuscus exhibit higher commercial value than males due to their larger size (resulting from faster growth), rounded abdomen, robust physique, and slow, graceful swimming behavior. In breeding production, male and female Cyprinus carpio var. rubrofuscus can be distinguished only by macroscopic indicators such as external appearance and body shape of the abdomen and cloaca at the adult stage. At present, technical means for distinguishing the sex of XX and XY male and female Cyprinus carpio var. rubrofuscus at the juvenile stage remain unavailable. Therefore, the development of sex-specific molecular markers applicable to Cyprinus carpio var. rubrofuscus possesses important application value for achieving early sex identification and mono-sex culture of ornamental Cyprinus carpio var. rubrofuscus, thereby improving cultivation efficiency.

### SUMMARY OF THE INVENTION

The object of the present invention is to provide a common carp sex-determining gene, which is a transcription factor isolated and cloned from Cyprinus carpio Huanghe var. The gene has not been previously reported in any research. Based on functional studies of the gene by the inventor, the gene has been designated ccMD (Cyprinus carpio Male Determinator) by the inventor. The protein encoded by the gene is shown in SEQ ID NO: 3.

The other object of the present invention is to provide the common carp sex-determining gene ccMD for its application in carp sex identification.

The further object of the present invention is to provide the common carp sex-determining gene ccMD for its application in carp sex control.

To achieve the above objects, the present invention provides the following technical solutions:

The inventor identified a Y chromosome-specific transcription factor from Cyprinus carpio Huanghe var. based on gene cloning technology. The gene has not been previously reported in any research. Based on functional studies of the gene by the inventor, the applicant has designated it ccMD, and its sequence structural characteristics are shown in FIGS. 1A and 1B. Open reading frame (ORF) predictive analysis revealed that the gene contains an open reading frame of 231 bp, encoding a low-molecular-weight protein of 76 amino acids with a molecular weight of 8.35 kDa. The protein contains an HLH (helix-loop-helix) domain, belongs to the bHLH superfamily, and is predicted to function as a transcription factor. The gene encodes the protein of SEQ ID NO: 3, where the full-length sequence of the gene is shown in SEQ ID NO: 1, and the coding sequence (CDS) is shown in SEQ ID NO: 2.

The inventors further analyzed the sequence and expression characteristics of ccMD in Cyprinus carpio Huanghe var., and demonstrated that ccMD is specifically present in the male genome of common carp and is specifically expressed at the early stage of testicular differentiation in common carp. Through chromosomal fluorescence in situ hybridization, the applicant demonstrated that the ccMD gene is localized on the Y chromosome of Cyprinus carpio Huanghe var., with 0, 1, and 2 copies of the ccMD gene present in XX, XY, and YY common carp, respectively. Based on The characteristic, quantitative PCR primers can be designed based on the sequences shown in SEQ ID NO: 1 or SEQ ID NO: 2, enabling rapid and accurate identification of XX, XY, and YY common carp at the juvenile stage. Furthermore, reagents for detecting the ccMD gene can achieve sex identification of XX and XY common carp.

Therefore, reagents for detecting genes encoding the protein shown in SEQ ID NO: 3, including but not limited to primers and DNA probes, also fall within the scope of protection of the present invention.

The application of a reagent for detecting a gene encoding the protein shown in SEQ ID NO: 3 for sex identification of common carp also falls within the scope of protection of the present invention.

For the aforementioned applications, the primers are preferably ccMD-rtF1:5′—

(SEQ ID NO: 11)
AGAACATGCTCTGGCAAACG-3′
and
ccMD-rtR1:
(SEQ ID NO: 12)
5′-TTTAGGGTTCACACCTCAGCG-3′

Among the above applications, it is preferred that the reagents can be used for sex identification of ornamental Cyprinus carpio var. rubrofuscus.

The application of the common carp sex-determining gene ccMD in carp sex control comprises: knocking out the ccMD gene in XY male common carp by a molecular biology method, such that the protein encoded by the ccMD gene loses its native activity, and gonadal development of the resulting gene-knockout common carp is completely reversed from testis to ovary; or overexpressing the ccMD gene in XX female common carp by a molecular biology method, such that gonadal development of the resulting transgenic adult fish is completely reversed from ovary to testis.

In the aforementioned applications, the carp is preferably selected from Cyprinus carpio Huanghe var., Cyprinus carpio var. singuonensis, Cyprinus carpio var. wuyuanensis, Cyprinus carpio carpio, Cyprinus carpio var. rubrofuscus, and/or Cyprinus acutidorsalis.

In the aforementioned applications, when the ccMD gene is knocked out, it is preferred that a key region encoding the protein of the ccMD gene in XY genetic male common carp be mutated or deleted by CRISPR/Cas9 gene editing technology.

In the aforementioned applications, it is preferred that the resulting knockout XY genetic male common carp comprise the sequence shown in SEQ ID NO: 4 or SEQ ID NO: 6.

In the aforementioned applications, when the ccMD gene is overexpressed, it is preferred that a ccMD gene overexpression vector be constructed, and the ccMD gene be introduced into XX genetic female common carp by gene transfer technology for expression.

In the aforementioned applications, it is preferred that the resulting ccMD overexpressed XX genetic female common carp comprise the sequence shown in SEQ ID NO: 5.

Compared with the prior art, the present invention possesses the following advantages.

For the first time, the applicant isolated and cloned the common carp sex-determining gene ccMD from Cyprinus carpio. The protein encoded by the gene is shown in SEQ ID NO: 3, and the corresponding polynucleotide in common carp is shown in SEQ ID NO: 1.

By utilizing the sequence features of the gene to design specific primers, XX, XY and YY common carp can be rapidly and accurately identified.

With the gene serving as a target gene, genetically male but physiologically female common carp, or genetically female but physiologically male common carp, can be rapidly obtained by means of gene editing technology and gene transfer technology without hormone treatment.

The present invention provides an accurate genetic target for sex identification and sex-controlled breeding of common carp, and provides novel genetic resources for superior germplasm creation in common carp aquaculture and ornamental breeding, as well as for the prevention and control of common carp as invasive alien species.

### BRIEF DESCRIPTION OF THE DRAWINGS

FIG. 1A illustrates the full-length mRNA sequence of the ccMD gene (SEQ ID NO: 33), and light gray shading indicates the positions of the start codon and stop codon; FIG. 1B illustrates the protein sequence encoded by the ccMD gene (SEQ ID NO: 3), and the sequence of the helix-loop-helix domain in the ccMD protein is indicated by a box; FIG. 1C is a schematic diagram of the ccMD gene structure.

FIG. 2A presents the results of gene amplification and identification of ccMD in 48 male and 48 female wild common carp populations from Hubei; FIG. 2B presents the gene amplification results of ccMD in 71 all-female common carp populations from Hubei, wherein PC denotes the positive control; FIG. 2C presents the gene identification results of ccMD in 48 male and 48 female wild common carp populations from Anhui; FIG. 2D presents the gene amplification results of ccMD in 18 male and 24 female wild common carp populations from Henan, wherein NC denotes the negative control.

FIG. 3A illustrates the chromosomal fluorescence in situ hybridization results of the Cyprinus carpio Huanghe var. ccMD gene; FIG. 3B illustrates the quantitative fluorescence PCR results of the Cyprinus carpio Huanghe var. ccMD gene, wherein the vertical axis represents relative DNA copy number; FIG. 3C illustrates the spatial and tissue-specific expression levels of Cyprinus carpio Huanghe var. ccMD gene in male and female common carp during early gonadal differentiation, wherein the vertical axis represents relative expression level.

FIG. 4A is a schematic diagram illustrating the construction of the homozygous line of Cyprinus carpio Huanghe var. ccMD gene mutation; FIG. 4B is a schematic diagram illustrating the construction and passage of the homozygous line of Cyprinus carpio Huanghe var. ccMD gene mutation.

FIG. 5A is a schematic diagram illustrating PCR identification and analysis of the genotype of the Cyprinus carpio Huanghe var. ccMD gene mutant homozygous line; FIG. 5B shows the predicted protein structure of the mutated ccMD in Cyprinus carpio Huanghe var.; FIG. 5C is a schematic diagram illustrating quantitative analysis of time-series expression of the ccMD gene in the Cyprinus carpio Huanghe var. ccMD gene mutant homozygous line, wherein the vertical axis represents the relative expression fold change of the mutant YY-288 ccMD gene; FIG. 5D presents the time-series histological section results of gonads from the Cyprinus carpio Huanghe var. ccMD gene mutant homozygous line.

FIG. 6A is a schematic diagram illustrating the construction of a homozygous line overexpressing the Cyprinus carpio Huanghe var. ccMD gene; FIG. 6B is a schematic diagram illustrating the construction and passage of homozygous lines overexpressing the Cyprinus carpio Huanghe var. ccMD gene.

FIG. 7A illustrates PCR identification of the genotype of the Cyprinus carpio Huanghe var. ccMD gene overexpression homozygous line; FIG. 7B presents an analysis of the expression level of the ccMD gene in the Cyprinus carpio Huanghe var. overexpression homozygous line, wherein the vertical axis represents relative gene expression level; FIG. 7C presents the results of gonadal anatomy and histological section of adult Cyprinus carpio Huanghe var.; FIG. 7D presents the expression levels of genes related to male and female sex differentiation in adult Cyprinus carpio Huanghe var., wherein the vertical axis represents relative gene expression level.

FIG. 8A presents the amplification and identification analysis of specific primers of the Cyprinus carpio Huanghe var. ccMD gene in male and female populations of Cyprinus carpio var. singuonensis; FIG. 8B presents the amplification and identification analysis of Cyprinus carpio Huanghe var. ccMD gene-specific primers in male and female populations of Cyprinus carpio var. wuyuanensis; FIG. 8C presents the amplification and identification analysis of Cyprinus carpio Huanghe var. ccMD gene-specific primers in male and female populations of Cyprinus carpio carpio; FIG. 8D presents the amplification and identification analysis of specific primers of the Cyprinus carpio Huanghe var. ccMD gene in male and female populations of Cyprinus carpio var. rubrofuscus; FIG. 8E presents the amplification and identification analysis of specific primers of the Cyprinus carpio Huanghe var. ccMD gene in male and female populations of Cyprinus acutidorsalis; FIG. 8F illustrates the homozygous line phenotype of the Cyprinus carpio var. singuonensis ccMD gene mutation; FIG. 8G presents the P₀ generation overexpression results of the ccMD gene in Cyprinus carpio var. singuonensis and Cyprinus carpio var. rubrofuscus.

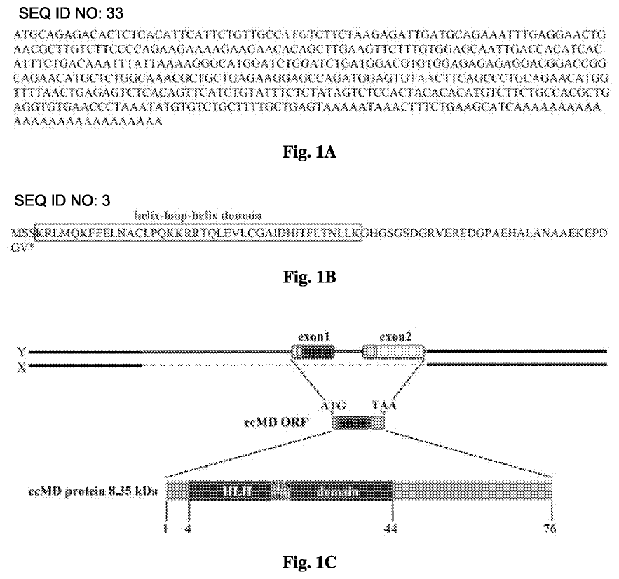

### DETAILED DESCRIPTION OF THE INVENTION

Hereinafter, the present invention will be described in detail with reference to specific embodiments. Based on the following description and embodiments, those skilled in the art can determine the essential features of the invention and can make various changes and modifications thereto to adapt it for various uses and conditions without departing from the spirit and scope of the invention. After prolonged natural differentiation and artificial selection, common carp has formed a series of local populations and varieties exhibiting diverse morphologies, rich body coloration, scaled and scaleless phenotypes, and strong adaptability to specialized environments. Mainstream common carp strains are mostly designated according to their geographic distribution and morphological characteristics. Although numerous strains exist, traditional morphological studies and modern molecular biological analyses have demonstrated that all common carp populations or strains belong to the same species from a taxonomic perspective. In the present application, the applicant collected common carp populations or strains distributed in different regions of China for verification, and demonstrated that the gene sequence of the present invention is conserved and universally applicable to all common carp of the genus Cyprinus.

### Embodiment 1

Acquisition of the ccMD gene and male-specific identification in Cyprinus carpio Huanghe var.

The whole genome of one XY male common carp with defined genetic background was sequenced using the PacBio Sequel platform, and the whole genomes of a full-sibling male and female common carp population (50 females and 50 males) obtained by test cross between XY male common carp and XX female common carp were resequenced using the HiSeq 4000 platform. A total of 606 candidate male-specific sequences with an aggregate length of approximately 14.7 Mb were obtained through initial screening by bioinformatics comparative analysis. Using the candidate sequence pool as template, primers were designed individually and subjected to PCR amplification in male and female common carp DNA. Ultimately, a pair of primers was successfully identified that yielded PCR amplification (BS14-F1:5′TGGTATCGATTTGATGACTCTGTAG-3′ (SEQ ID NO: 7) and BS15-R1:5′CTTCATTGTGCAGATGCCTACATTA-3′ (SEQ ID NO: 8)), and a fragment exhibiting length polymorphism between X and Y chromosomes was successfully obtained. A male-specific fragment with a length of 1487 bp was obtained by cloning and sequencing (as shown in SEQ ID NO:1).

A male-specific gene comprising two exons was successfully identified within the 1487 bp sequence by transcriptome annotation and RACE-PCR full-length cloning technology. Due to the absence of prior functional reports, it was designated ccMD. Specifically, the full-length ccMD gene was obtained by amplification; the polynucleotide sequence is shown in SEQ ID NO:1, the coding sequence (CDS) is shown in SEQ ID NO: 2, and the encoded protein is shown in SEQ ID NO:3. The gene contains an open reading frame of 231 bp, encoding a low-molecular-weight protein of 76 amino acids with a molecular weight of 8.35 kDa. The protein contains an HLH (helix-loop-helix) domain, belongs to the bHLH superfamily, and is predicted to function as a transcription factor.

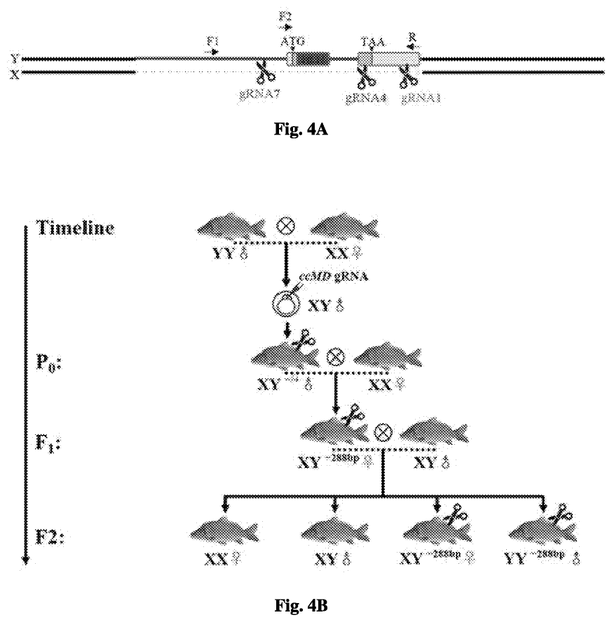

DNA from Cyprinus carpio Huanghe var. with diverse genetic backgrounds collected from Hubei, Anhui, Henan, and other regions was used as template, and routine Taq polymerase was employed for PCR amplification and identification. The amplification system is shown in Table 1, and the amplification procedure is shown in Table 2. The primers used were ccMD gene-specific primers: F1:5′-AACTGCTAGTTGGTCAGACTTGCGA-3′ (SEQ ID NO: 9) and R: 5′-CACATATTTAGGGTTCACACCTCAG-3′ (SEQ ID NO: 10) (see FIG. 4A for primer design positions).

TABLE 1
Amplification System for ccMD Gene-Specific Primers

| Reagent | Volume | |
|---|---|---|
| 2x Rapid Taq Master Mix | 10 | μL |
| Forward primer/reverse primer | 0.5/0.5 | μL |
| Template DNA | 1 | μL |

| | |
|---|---|
| ddH₂O | up to 20 μL |

TABLE 2
PCR Amplification Protocol for ccMD
Gene-Specific Primer Identification

| | | | | | Number |
|---|---|---|---|---|---|
| Protocol | Temperature | Time | | | of Cycles |
| Pre-denaturation | 95° C. | 2 | min | | |
| Denaturation | 95° C. | 15 | sec | | |
| Annealing | 60° C. | 15 | sec | | |
| Extension | 72° C. | 20 | sec | {close oversize brace} | 35 |
| Final extension | 72° C. | 2 | min | | |

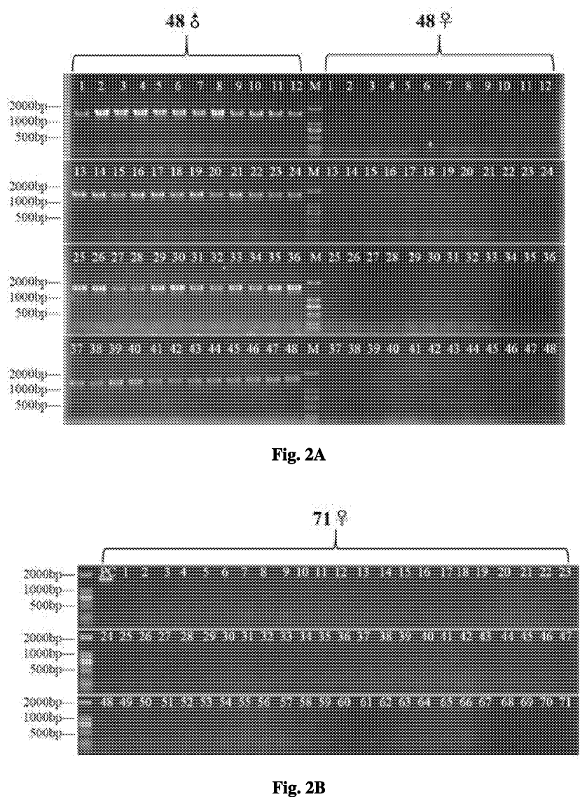

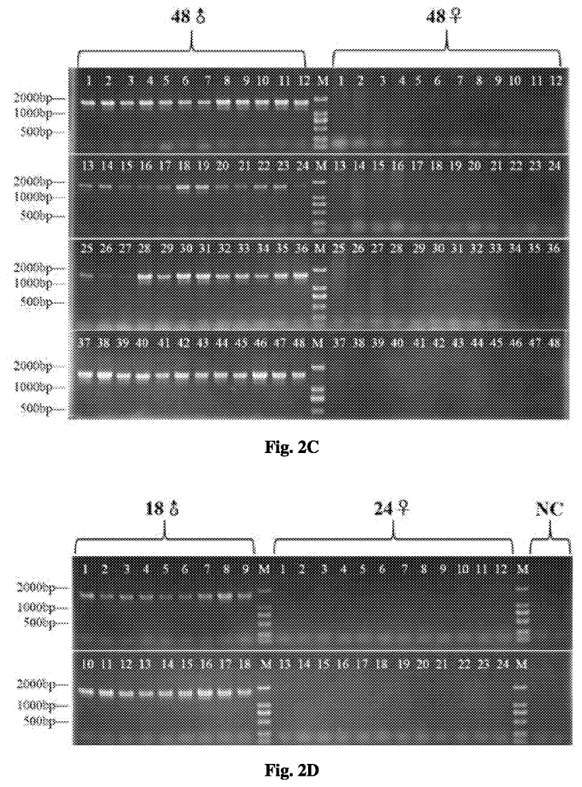

The results of agarose gel electrophoresis demonstrated that ccMD gene-specific primers exhibited male-specific amplification patterns in 48 male and 48 female wild populations from Hubei (FIG. 2A), 71 all-female populations from Hubei (FIG. 2B), 48 male and 48 female wild populations from Anhui (FIG. 2C), and 18 male and 24 female wild populations from Henan (FIG. 2D); specifically, bands were amplified in males but absent in females. These results demonstrate that the DNA sequence of the ccMD gene is male-specific and yields consistent results across multi-background common carp populations, indicating broad applicability.

### Embodiment 2

Chromosomal localization as well as spatial and tissue-specific expression pattern analysis of Cyprinus carpio Huanghe var. ccMD gene

A digoxigenin-labeled specific DNA probe was prepared using the 1487 bp male-specific DNA sequence (segment indicated in FIG. 4A) harboring the ccMD gene as template; the specific DNA probe sequence is shown in SEQ ID NO:1. The probe sequence of the present invention is completely identical to the 1487 bp male-specific DNA sequence.

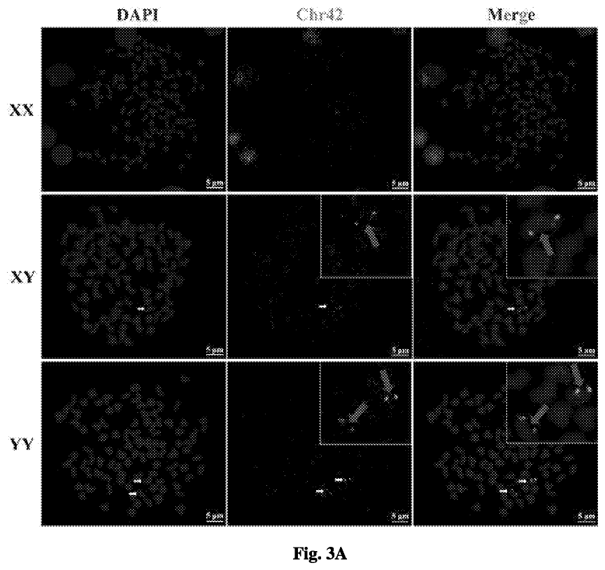

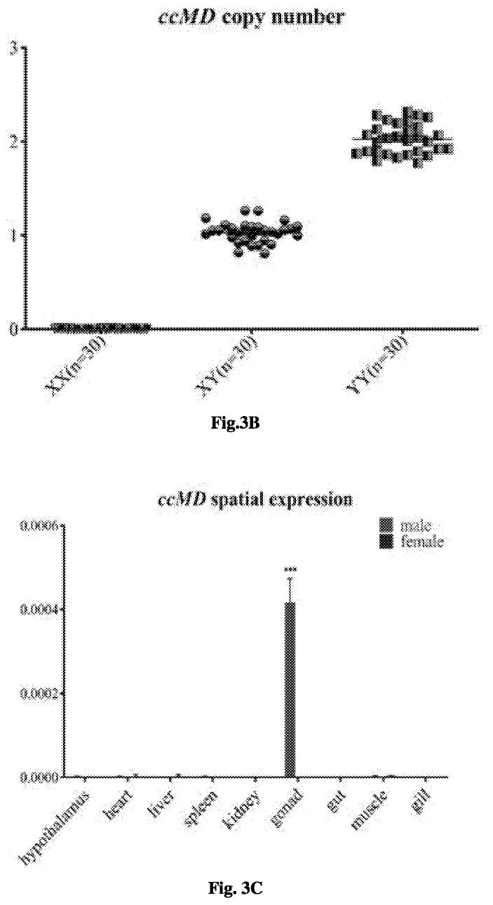

Chromosomal fluorescence in situ hybridization analysis was performed on metaphase chromosomes prepared from XX, XY, and YY common carp. The results demonstrated that the specific probes produced signals on 0, 1, and 2 metaphase chromosomes in XX, XY, and YY common carp, respectively (FIG. 3A). The metaphase chromosomes exhibiting probe localization in XY and YY common carp were consistent in morphology, all exhibiting telocentric characteristics, indicating that the Y chromosome of Cyprinus carpio Huanghe var. is a telocentric chromosome, and the ccMD gene is located at the terminal position of the Y chromosome distal to the centromere.

The copy number of the ccMD gene was quantitatively determined using XX, XY, and YY Cyprinus carpio Huanghe var. as DNA templates and the sGnRH gene, which maintains constant copy number in Cyprinus carpio Huanghe var., as internal reference. The quantitative primers for the ccMD gene were as follows: ccMD-rtF1:5′-AGAACATGCTCTGGCAAACG-3′ (SEQ ID NO: 11) and ccMD-rtR1:5′-TTTAGGGTTCACACCTCAGCG-3′ (SEQ ID NO: 12); the internal reference sGnRH gene quantification primers were: sGnRH-rtF1:5′-ATGGAGTGGAACGGAAGGT-3′ (SEQ ID NO: 13) and sGnRH-rtR1:5′-CAGACAAATGAGAACAAGAGGAA-3′ (SEQ ID NO: 14). Consistent with the results of chromosomal fluorescence in situ hybridization, the quantitative PCR (qPCR) results also demonstrated that the copy number of the ccMD gene in YY common carp was approximately twice that in XY common carp (FIG. 3B), further indicating that the ccMD gene is a Y chromosome-specific gene of Cyprinus carpio Huanghe var., which can be used for rapid identification of XX, XY, and YY common carp at the juvenile stage.

The β-actin gene was used as the internal reference gene, and cDNA derived from hypothalamus, heart, liver, spleen, kidney, gonad (testis in male common carp, ovary in female common carp), intestine, muscle, and gill tissues of male and female common carp at the gonadal differentiation stage was used as template. The tissue-specific expression pattern of ccMD was analyzed by qPCR. The qPCR primers used for the internal reference β-actin gene were: β-actin-rtF: 5′-GATGATGAAATTGCCGCACTG-3′ (SEQ ID NO: 15) and β-actin-rtR:5′-ACCAACCATGACACCCTGATGT-3′ (SEQ ID NO: 16); the qPCR primers for the ccMD gene were: ccMD-rtF: 5′-TGACAAATTTATTAAAAAGGCATGG-3′ (SEQ ID NO: 17) and ccMD-rtR:5′-CACATATTTAGGGTTCACACCTCAG-3′ (SEQ ID NO: 18). Spatial and tissue-specific fluorescence quantification results demonstrated that ccMD exhibited spatial and tissue-specific expression patterns and was exclusively expressed in male common carp gonads (testis) during sex differentiation (FIG. 3C).

### Embodiment 3

Cyprinus carpio Huanghe Var. ccMD Gene Knockout and Phenotypic Analysis
1) Design of gRNA Targets for ccMD Gene Editing

Using the ccMD gene open reading frame (ORF) sequence as template, three highly efficient gRNA sequences targeting the coding region of the ccMD protein and the upstream and downstream non-coding regions (see FIG. 4A for the schematic diagram of target design) were designed using the ZiFiT gene editing target online prediction tool (http://zifit.partners.org/ZiFiT/CSquare9Nuclease.aspx), designated gRNA7 (target sequence: GGCCAGAGCAGAAATGTTG (SEQ ID NO: 19)), gRNA4 (target sequence: GGACGTGTGGAGAGAGAGGA (SEQ ID NO: 20)), and gRNA1 (target sequence: GGCAGAAGACATGTGTGTAG (SEQ ID NO: 21)), respectively.

The gRNA amplification primers used were: T7-gRNA7-F:5′—

(SEQ ID NO: 22)
TGTAATACGACTCACTATAggccagagcagaaatgttgGTTTTAGAGCT
AGAAAT-3′,
T7-gRNA4-F:
(SEQ ID NO: 23)
5′-TGTAATACGACTCACTATAggacgtgtggagagagaggaGTTTTAG
AGCTAGAAAT-3′,
T7-gRNA1-F:
(SEQ ID NO: 24)
5′-TGTAATACGACTCACTATAggcagaagacatgtgtgtagGTTTTAG
AGCTAGAAAT-3′,
and
gRNA-RP:
(SEQ ID NO: 25)
5′-AAAAAAAGCACCGACTCGGTGCCAC-3′.

Mixed-target injection of gRNA7, gRNA4, and gRNA1 was subsequently performed to efficiently obtain P₀ generation positive common carp with complete loss of ccMD gene function.

2) gRNA Synthesis

The DNA templates required for in vitro transcription of gRNA were obtained by PCR amplification, and in vitro gRNA transcription was performed using the MEGAshortscript™ Transcription Kit (Ambion, USA). The in vitro transcribed gRNA was purified using the mir Vana™ miRNA Isolation Kit (Ambion, USA). The final injection mixture was prepared on ice, comprising 500 ng/μL TrueCut™ Cas9 Protein v2 (Invitrogen, USA), 50 ng/μL ccMD gRNA, and 10% (v/v) Phenol Red (Sigma-Aldrich, USA), thoroughly mixed, and maintained at the appropriate temperature pending use.

3) Development of ccMD Gene Knockout Common Carp

Male XY embryos of Cyprinus carpio Huanghe var. were obtained by crossing YY Cyprinus carpio Huanghe var. (Jiang et al., 2018) as male parent with common XX Cyprinus carpio Huanghe var. as female parent. Using a microinjector (Eppendorf, Germany), the prepared ccMD gRNA, CAS9 protein, and Phenol Red were uniformly mixed and introduced into one-cell stage XY all-male Cyprinus carpio Huanghe var. embryos by microinjection to obtain XY common carp with knockout of the ccMD gene.

The obtained ccMD gene knockout common carp were propagated to establish a pedigree using conventional methods for constructing fish CRISPR/Cas9 gene editing pedigrees, and ccMD gene homozygous mutant common carp were obtained in the F2 generation (FIG. 4B).

4) Phenotypic Analysis of ccMD Knockout Common Carp

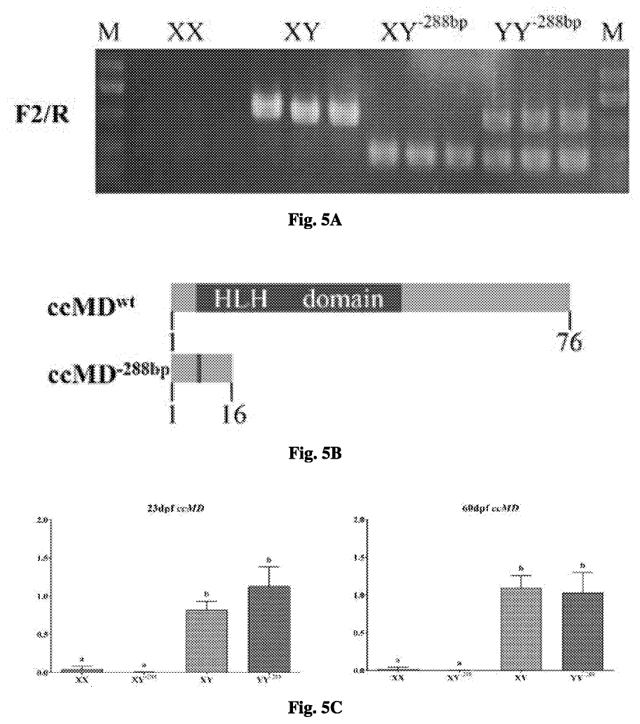

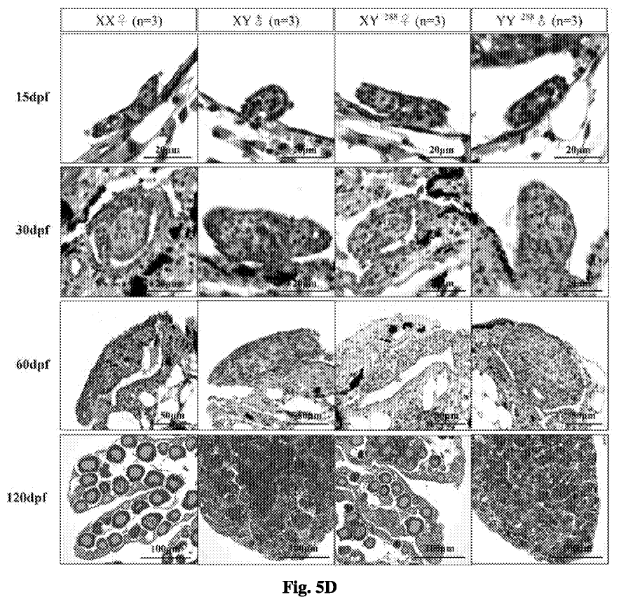

Identification was performed using primers F2/R (FIG. 4A; F2 primer sequence: 5′-CACATATTTAGGGTTCACACCTCAG-3′ (SEQ ID NO: 26); R primer sequence as shown in Embodiment 1). Homozygous families of ccMD mutant F2 were identified by PCR amplification (FIG. 5A). The ccMD mutants XY-288 and YY-288 (YY-288 harbored mutation in only one ccMD copy and theoretically still developed into male common carp) were obtained, and common carp with a 288 bp deletion in the ccMD gene contained the sequence shown in SEQ ID NO:4. The coding situation of the XY-288 mutant homozygous ccMD protein was predicted (FIG. 5B). The results demonstrated that, compared with the non-specific main band in XX, XY, XY-288, and YY-288 target amplification exhibited one large band, one small main band, and one large and one small main band, respectively. The XY-288 homozygous ccMD-encoded protein exhibited large segment deletion and frameshift. Function failure was predicted. Therefore, mutation of the ccMD gene such that the ccMD-encoded protein loses functionality can enable the transformation of common carp from male to female.

Real-time fluorescence quantitative analysis of ccMD expression in the gonadal development time series of homozygous families was performed (FIG. 5C). Quantitative results at key gonadal developmental time points of 23 dpf and 60 dpf demonstrated that ccMD gene expression levels were consistent between XY and YY-288, and between XX and XY-288 The expression in XY and YY-288 genotype individuals was significantly higher than that in XX and XY-288 genotype individuals at each time point, indicating that the constructed F₂ generation ccMD gene mutation homozygous family was effective.

The gonadal developmental phenotype of homozygous families was analyzed by histological sectioning. Hematoxylin and eosin (H & E) staining results (FIG. 5D) demonstrated that prior to sex determination at 15 dpf, no obvious differences in gonadal structure were observed among XY, XY⁻²⁸⁸, and YY⁻²⁸⁸ common carp, consisting primarily of gonadal somatic cells. At the critical period of sex determination at 30 dpf, gonads of the above types began to exhibit differences. The gonadal structure of XY⁻²⁸⁸ was consistent with that of XX common carp, and the ovarian cavity structure unique to females appeared, whereas the gonads of XY and YY⁻²⁸⁸ common carp lacked The structure. Subsequently, at the critical periods of sexual differentiation at 60 dpf and 120 dpf, XY⁻²⁸⁸ remained consistent with the gonadal structure of XX common carp, with enlarged ovarian cavity and continuous proliferation and growth of oocytes, whereas XY and YY-288 were consistent with each other, gradually entering the spermatogenesis process exhibiting typical masculinization characteristics. Collectively, these results indicate that following ccMD mutation, XY⁻²⁸⁸ mutant homozygotes exhibit typical feminization developmental characteristics at all stages of gonadal development, demonstrating that the ccMD gene is essential for male sex determination in Cyprinus carpio Huanghe var.

### Embodiment 4

Overexpression and Phenotypic Analysis of ccMD Gene in Cyprinus carpio Huanghe var.

### 1) Overexpression Vector Construction:

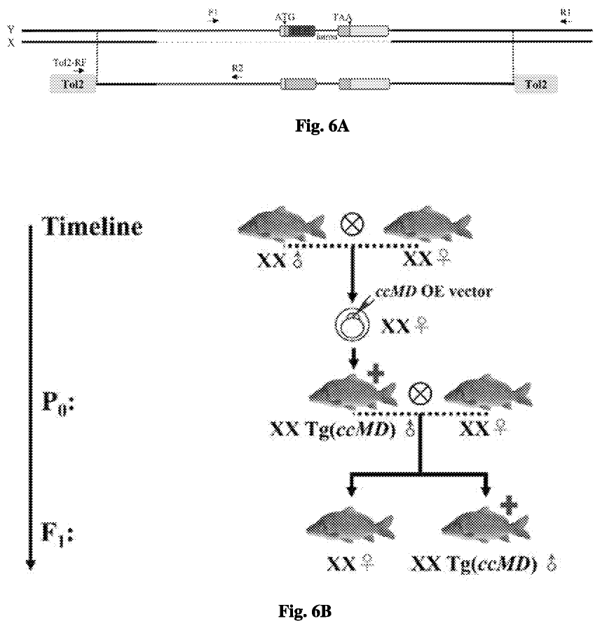

The ccMD overexpression vector was constructed based on the pSK_MCS_EGFP_Tol2 vector (Xia et al., 2018). Specifically, the pSK_MCS_EGFP_Tol2 plasmid was first double-digested with EcoRI and XbaI, and subsequently a 3 kb sequence containing the complete gene structure of the ccMD gene (as shown in SEQ ID NO:5) was ligated by homologous recombination (see FIG. 6A for the schematic diagram of vector construction). The ccMD overexpression gene fragment was cloned using XY male common carp DNA as template, and the PCR amplification primers were: EcoRI-BS14—

F1:
(SEQ ID NO: 27)
5′-TCGATAAGCTTGATATCGTGGTATCGATTTGATGACTCTGTAG-3′,
and
XbaI-BS15-R1:
(SEQ ID NO: 28)
5′-ATACGACTCACTATAGTTCTTCATTGTGCAGATGCCTACATTA-3′

2) Development of ccMD Overexpressed Common Carp

An injection mixture was prepared comprising linearized overexpression plasmid vector at a final concentration of 50 ng/μL, Tol2 mRNA at 100 ng/μL, and Phenol Red at 10% (v/v), thoroughly mixed, and maintained for subsequent use. XX pseudo-male common carp (Jiang et al., 2020) was used as the male parent and common XX Cyprinus carpio Huanghe var. as the female parent to produce XX Cyprinus carpio Huanghe var. offspring by crossing. The mixed ccMD overexpression vector solution was microinjected into one-cell stage XX all-female Cyprinus carpio Huanghe var. embryos by microinjection technique to obtain XX common carp with overexpression of the ccMD gene. The ccMD overexpressed common carp homozygous families were obtained in the F₁ generation by serial propagation using methods conventional in the art for establishing transgenic fish pedigrees (FIG. 6B).

3) Phenotypic Analysis of ccMD Gene Overexpression Common Carp

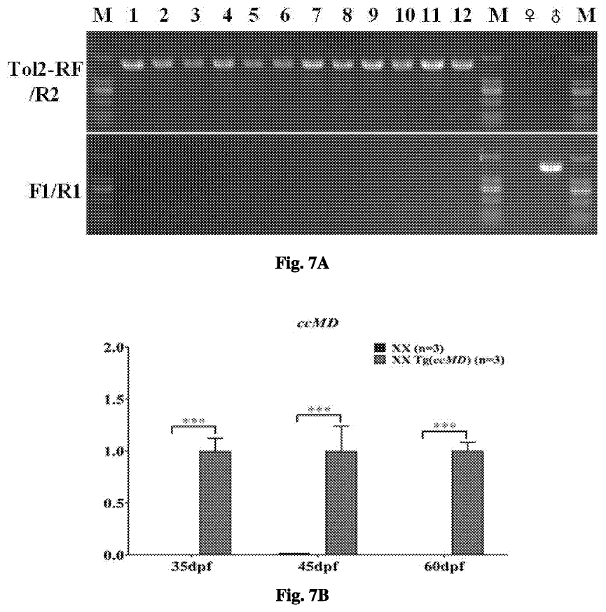

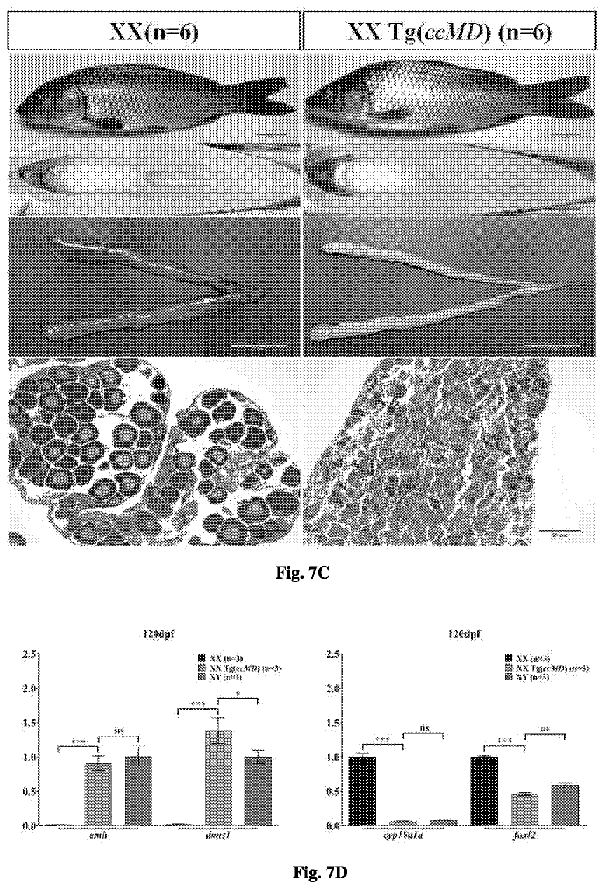

The ccMD gene overexpression families were identified by PCR genotyping and real-time fluorescence quantitative PCR (FIG. 7A, FIG. 7B), using hereditary-specific identification primers (forward primer F1:5′-AACTGCTAGTTGGTCAGACTTGCGA-3′ (SEQ ID NO: 29), reverse primer R1:5′-ATGAACTGGATTTTCTGCAACACTG-3′ (SEQ ID NO: 30); primer design shown in FIG. 6A) and overexpression-specific identification primers (forward primer Tol2-RF: 5′-AAAGTATCTGGCTAGAATCTTACT-3′ (SEQ ID NO: 31), reverse primer R2:5′-TGTTGAGTGACTGGTGAGGTGAAGG-3′ (SEQ ID NO: 32); primer design shown in FIG. 6A). The results demonstrated that the overexpressed F₁ generation family indeed comprised two genotypes: wild-type XX and ccMD gene overexpression homozygous XX/Tg (ccMD). Time-series fluorescence quantitative analysis results of the ccMD gene demonstrated that in homozygous families at 35 dpf, 45 dpf, and 60 dpf, the ccMD gene expression level in overexpression homozygous XX/Tg (ccMD) individuals was significantly higher than that in wild-type XX genotype individuals, indicating that F₁ generation homozygous family construction was effective.

The gonadal developmental phenotype of 120 dpf overexpression homozygous families was analyzed by gonadal anatomy and histological sectioning (FIG. 7C). The results demonstrated that the gonads of wild-type XX common carp appeared translucent and ovary-like, with sections filled with primary growth stage oocytes, whereas the gonads of XX/Tg (ccMD) common carp appeared gray-white and striated, with sections composed of male germ cells at various meiotic stages, exhibiting typical male gonadal developmental characteristics. Quantitative analysis of male sex differentiation-related genes amh and dmrt1, and female sex differentiation-related genes cyp19a1a and foxl2, in 120 dpf overexpressed families was performed (FIG. 7D). The results demonstrated that XX/Tg (ccMD) overexpression homozygotes also exhibited obvious masculinization characteristics at the gene expression level, which was highly consistent with wild-type XY common carp.

The above results obtained in Cyprinus carpio Huanghe var. demonstrated that ccMD is specifically present in the male genome of common carp and is specifically expressed at the early stage of testicular differentiation. Knockout of ccMD in XY common carp caused XY common carp to develop into females. Introduction of ccMD expression in XX female common carp led to complete reversal of gonadal developmental fate from female to male. Collectively, these results demonstrate that ccMD is the male sex-determining gene of Cyprinus carpio Huanghe var.

### Embodiment 5

Conservation of the Cyprinus carpio Huanghe var ccMD gene in freshwater fish species of the genus Cyprinus:

To investigate the conservation of sex-determining genes among different geographical strains or subspecies of common carp, studies were conducted in Cyprinus carpio var. singuonensis, Cyprinus carpio var. wuyuanensis, Cyprinus carpio carpio, Cyprinus carpio var. rubrofuscus, and Cyprinus acutidorsalis.

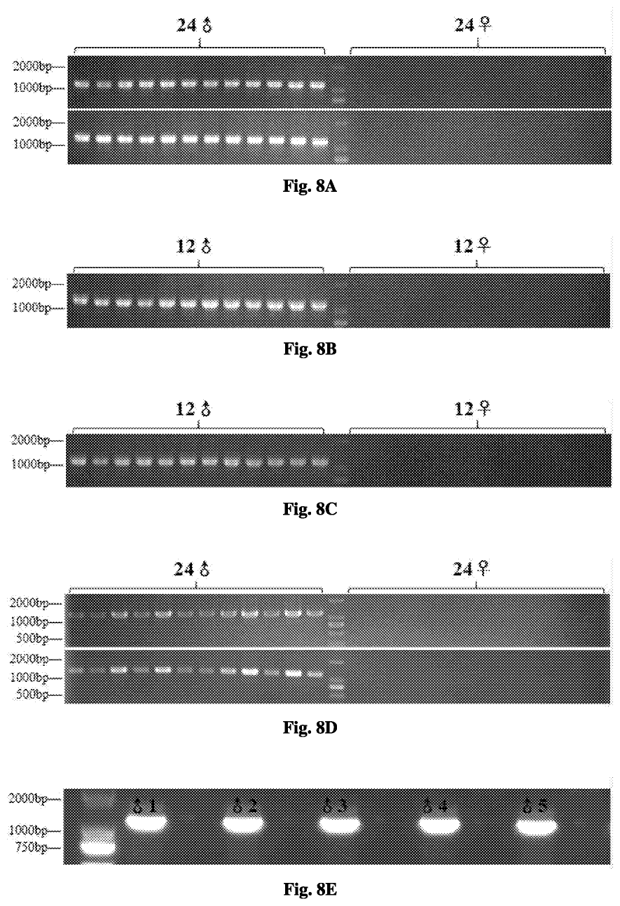

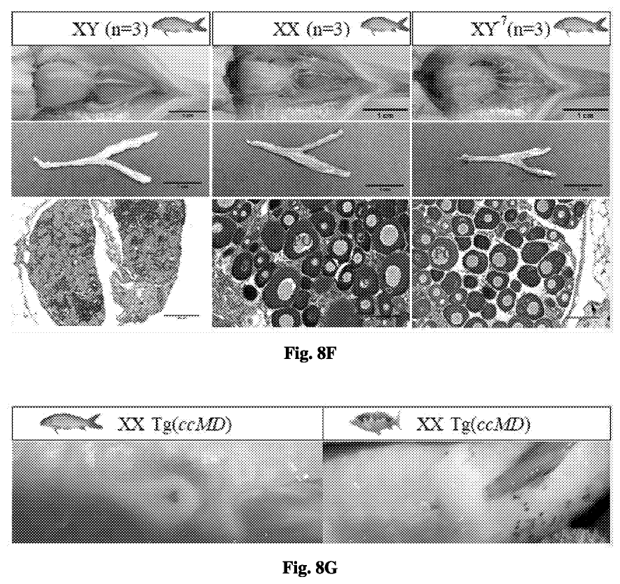

Using DNA from wild male and female populations of Cyprinus carpio var. singuonensis, Cyprinus carpio var. wuyuanensis, Cyprinus carpio carpio, Cyprinus carpio var. rubrofuscus, and Cyprinus acutidorsalis as templates, amplification and identification were sequentially performed using the gene-specific primer combination F1/R of Cyprinus carpio Huanghe var. ccMD. The amplification results demonstrated obvious male specificity, indicating that the Cyprinus carpio Huanghe var. ccMD gene-specific primer combination can also accurately identify the sex of Cyprinus carpio var. singuonensis (FIG. 8A), Cyprinus carpio var. wuyuanensis (FIG. 8B), Cyprinus carpio carpio (FIG. 8C), Cyprinus carpio var. rubrofuscus (FIG. 8D), and Cyprinus acutidorsalis (FIG. 8E).

Similar to the method applied in Cyprinus carpio Huanghe var., CRISPR/Cas9 gene editing technology and subsequent propagation were used to construct the ccMD mutant homozygous family of Cyprinus carpio var. singuonensis-7 bp (The family contains the sequence shown in SEQ ID NO: 6). It was predicted that the deletion protein lost its native function. Anatomical and histological sectioning results of the gonads of 4-month-old adult fish demonstrated (FIG. 8F) that, compared with the gray-white striated appearance and typical spermatogenesis-like testicular structure of wild-type XY male common carp, the XY-7 ccMD mutant homozygous gonads were consistent with those of wild-type XX female common carp, both exhibiting translucent striated morphology, and the sectioning results revealed typical ovarian structures filled with primary growth stage oocytes, indicating that the ccMD gene is essential for male sex determination in Cyprinus carpio var. singuonensis.

Using the ccMD overexpression vector consistent with that used in Cyprinus carpio Huanghe var., ccMD overexpression analysis was performed in Cyprinus carpio var. singuonensis and Cyprinus carpio var. rubrofuscus. Screening and identification were conducted using genetic sex identification primers and overexpression-specific identification primers, and XX/Tg (ccMD) genotype Cyprinus carpio var. singuonensis overexpression and XX/Tg (ccMD) genotype Cyprinus carpio var. rubrofuscus overexpression P₀ generation positive fish were obtained. Adult fish semen extrusion assessment results demonstrated (FIG. 8G) that P₀ generation positive fish of both genotypes produced semen, exhibiting typical male sexual characteristics. These results indicate that the ccMD gene is also sufficient to initiate male sex determination in Cyprinus carpio var. singuonensis and Cyprinus carpio var. rubrofuscus.

We claim:

1. A reagent for detecting ccMD gene, wherein an amino acid sequence of a protein encoded by ccMD gene is as shown in SEQ ID NO. 3.

2. The reagent according to claim 1, wherein the reagent is a primer or a DNA probe.

3. The reagent according to claim 2, wherein the reagent is a primer pair of
ccMD-rtF1:
5′-AGAACATGCTCTGGCAAACG-3′;
and
ccMD-rtR1:
5′-TTTAGGGTTCACACCTCAGCG-3′.

4. The reagent according to claim 2, wherein the reagent is a DNA probe, and the DNA probe is a polynucleotide labeled with digoxin and shown in SEQ ID NO. 1.

5. The reagent according to claim 1, wherein a polynucleotide sequence of the ccMD gene is SEQ ID NO. 1 or SEQ ID NO. 2.

6. A method for identifying a gender of common carp, comprising:
detecting whether a genome of a common carp to be tested contains the ccMD gene using the reagent according to claim 1,
determining that, when the ccMD gene is present, the common carp is a male and when the ccMD gene is absent, the common carp is a female.

7. A method for controlling sex of common carp, comprising:
altering gene expression of ccMD gene in a common carp, wherein the ccMD gene has a nucleotide sequence of SEQ ID NO: 1 or SEQ ID NO: 2, and
reversing gonadal development in the common carp with the altered gene expression.

8. The method according to claim 7, wherein the common carp is a XY male common carp and the gene expression of the ccMD gene is altered by knocking out the ccMD gene.

9. The method according to claim 8, further comprising:
causing a protein having an amino acid sequence of SEQ ID NO:3 encoded by the ccMD gene to lose native activity, and
completely reversing the gonadal development of the XY male common carp from testis to ovary.

10. The method according to claim 8, wherein the ccMD gene is knocked out by mutating or deleting a key region encoding a protein of amino acid sequence of SEQ ID NO: 3 by CRISPR/Cas9 gene editing.

11. The method according to claim 8, wherein the obtained ccMD knockout XY male carp comprises a DNA sequence of SEQ ID NO: 4 or SEQ ID NO: 6.

12. The method according to claim 7, wherein the common carp is XX female common carp, and the gene expression of the ccMD gene is altered by overexpressing the ccMD gene in XX female common carp.

13. The method according to claim 12, further comprising:
constructing a ccMD gene overexpression vector,
introducing the ccMD gene overexpress vector into the XX female common carp and overexpressing the ccMD gene by gene transfer, and
completely reverse the gonadal development of the XX female common carp from ovary to testis.

14. The method according to claim 12, wherein the XX female common carp with overexpressed ccMD gene comprises a DNA sequence of SEQ ID NO: 5.

15. The method according to claim 7, wherein the common carp is selected from Cyprinus carpio Huanghe var., Cyprinus carpio var. singuonensis, Cyprinus carpio var. wuyuanensis, Cyprinus carpio carpio, Cyprinus carpio var. rubrofuscus, and/or Cyprinus acutidorsalis.
